(19) United States
(12) Patent Application Publication
(10) Pub. No.: US 2026/0043314 A1
(43) Pub. Date: Feb. 12, 2026

(54) VOLUMETRIC GEOMETRY QUANTIFICATION OF NEAR-SURFACE CARBON CONTAINERS

(71) Applicant: SCHLUMBERGER TECHNOLOGY CORPORATION, Sugar Land, TX (US)

(72) Inventors: Vasudhaven SUDHAKAR, Sugar Land, TX (US); Bahaa A. SOLIMAN, Houston, TX (US); Tom DYE, Houston, TX (US); Hilary BRAWNER, Houston, TX (US)

(21) Appl. No.: 19/141,341

(22) PCT Filed: Jan. 17, 2024
(86) PCT No.: PCT/US2024/011775

§ 371 Date: Jun. 20, 2025

Related U.S. Application Data
(60) Provisional application No. 63/480,230, filed on Jan. 17, 2023.

(51) Int. Cl. E21B 41/00; E21B 49/02; G01N 33/24; G01V 20/00

(52) U.S. Cl. CPC E21B 41/0064; E21B 49/02; G01N 33/243; G01V 20/00

(57) ABSTRACT

A method for carbon sequestration includes receiving geophysical survey data. The method also includes building a three-dimensional (3D) model based upon the geophysical survey data. The method also includes calibrating the 3D model to produce a calibrated 3D model. The method also includes extracting a soil layer from the calibrated 3D model to produce an extracted soil layer. The method also includes designing a soil-sampling campaign based upon the extracted soil layer. The soil-sampling campaign includes a plurality of cores. The method also includes determining characteristics of the cores. The method also includes propagating the characteristics through the calibrated 3D model to produce a 3D property model.

### CROSS-REFERENCE TO RELATED APPLICATIONS

This application claims priority to U.S. Provisional Patent Application No. 63/480,230, filed on Jan. 17, 2023, the entirety of which is incorporated by reference.

### BACKGROUND

The voluntary marketplace for carbon offsets is rapidly growing. Direct air carbon capture technologies are expensive and difficult to scale, whereas nature-based solutions are scalable, but may have reliability concerns. Thus, it can be desirable to quantify the size and capacity of terrestrial “carbon containers,” including the near-subsurface component. Understanding the size and capacity of carbon containers, as well as the residency time of carbon therein, can facilitate measuring nature-based soil carbon sequestration.

Recent studies demonstrated that shallow soil (˜1 ft) is exposed to farming practices and climate effects that shrink the CO2 capacity. With going deeper, the effect of farming practice, and weather changes is reduced, thereby increasing the soil's full potential of carbon sequestration capacity. Current natural-based carbon sequestration practices focus on the top of the soil container, that is visible and relatively easy to map. There is a need to develop cost-effective ultra near surface mapping and modeling methodology to unlock the potential capacity of the soil.

### SUMMARY

A method for carbon sequestration is disclosed. The method includes receiving geophysical survey data. The method also includes building a three-dimensional (3D) model based upon the geophysical survey data. The method also includes calibrating the 3D model to produce a calibrated 3D model. The method also includes extracting a soil layer from the calibrated 3D model to produce an extracted soil layer. The method also includes designing a soil-sampling campaign based upon the extracted soil layer. The soil-sampling campaign includes a plurality of cores. The method also includes determining characteristics of the cores. The method also includes propagating the characteristics through the calibrated 3D model to produce a 3D property model.

A computing system is also disclosed. The computing system includes one or more processors and a memory system. The memory system includes one or more non-transitory computer-readable media storing instructions that, when executed by at least one of the one or more processors, cause the computing system to perform operations. The operations include receiving geophysical survey data. The geophysical survey data includes seismic data, ground-penetrating radar, electric resistivity tomography, or a combination thereof. The operations also include building a three-dimensional (3D) model based upon the geophysical survey data. The 3D model is built using surface wave analysis modeling and inversion (SWAMI). The 3D model includes topsoil and impermeable strata. The topsoil is above the impermeable strata. The operations also include calibrating the 3D model to produce a calibrated 3D model. The 3D model is calibrated using electric resistivity tomography (ERT), ground penetrating radar (GPR), water well data, or a combination thereof. The operations also include extracting a soil layer from the calibrated 3D model to produce an extracted soil layer. The soil layer includes the topsoil and the impermeable strata. The operations also include designing a soil-sampling campaign based upon the extracted soil layer. The soil-sampling campaign includes a plurality of cores. The operations also include determining characteristics of physical cores from a real-world subsurface. The physical cores correspond to the cores in the extracted soil layer of the calibrated 3D model. The characteristics include biochemical soil characteristics and physical soil characteristics. The biochemical soil characteristics include macro nutrient characteristics, micro soil nutrient characteristics, existing soil organic matter, or a combination thereof. The physical soil characteristics include a density of the soil. The operations also include propagating the characteristics through the calibrated 3D model to produce a 3D property model.

A non-transitory computer-readable medium is also disclosed. The medium stores instructions that, when executed by one or more processors of a computing system, cause the computing system to perform operations. The operations include receiving geophysical survey data. The geophysical survey data includes seismic data, ground-penetrating radar, and electric resistivity tomography. The operations also include building a three-dimensional (3D) model based upon the geophysical survey data. The 3D model is built using surface wave analysis modeling and inversion (SWAMI). The 3D model includes topsoil and impermeable strata. The topsoil is above the impermeable strata. The operations also include calibrating the 3D model to produce a calibrated 3D model. The 3D model is calibrated using electric resistivity tomography (ERT), ground penetrating radar (GPR), and water well data. The operations also include extracting a soil layer from the calibrated 3D model to produce an extracted soil layer. The extracted soil layer includes the topsoil and the impermeable strata. The operations also include designing a soil-sampling campaign based upon the extracted soil layer. The soil-sampling campaign includes a plurality of physical cores that are to be collected from a real-world subsurface. The operations also include determining characteristics of the physical cores. The characteristics include biochemical soil characteristics and physical soil characteristics. The biochemical soil characteristics include macro nutrient characteristics, micro soil nutrient characteristics, and existing soil organic matter. The physical soil characteristics include a density of the soil. The operations also include propagating the characteristics through the calibrated 3D model to produce a 3D property model. The operations also include determining an effective available volume in the extracted soil layer of the 3D property model. The effective available volume is a carbon container. The operations also include determining a total value of a carbon sink in the carbon container. The total value is determined based upon the effective available volume. The operations also include determining a land management practice for sequestrating carbon in the carbon container. The land management practice is determined based upon the 3D property model and the total value of the carbon sink.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1 illustrates an example of a system that includes various management components to manage various aspects of a geologic environment, according to an embodiment.

FIG. 2 illustrates a flowchart of a method for carbon sequestration, according to an embodiment.

FIG. 3 illustrates a perspective view of geophysical survey data, according to an embodiment.

FIG. 4 illustrates a perspective view of a 3D model, according to an embodiment.

FIG. 5A illustrates a perspective view of deep soil core samples in the field, and FIG. 5B illustrates the core samples after being collected, according to an embodiment.

FIG. 6 illustrates a perspective view of the effective available volume (i.e., the potential carbon storage container), according to an embodiment.

FIG. 7 illustrates a schematic view of a computing system for performing at least a portion of the method, according to an embodiment.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

Attention is now directed to processing procedures, methods, techniques, and workflows that are in accordance with some embodiments. Some operations in the processing procedures, methods, techniques, and workflows disclosed herein may be combined and/or the order of some operations may be changed.

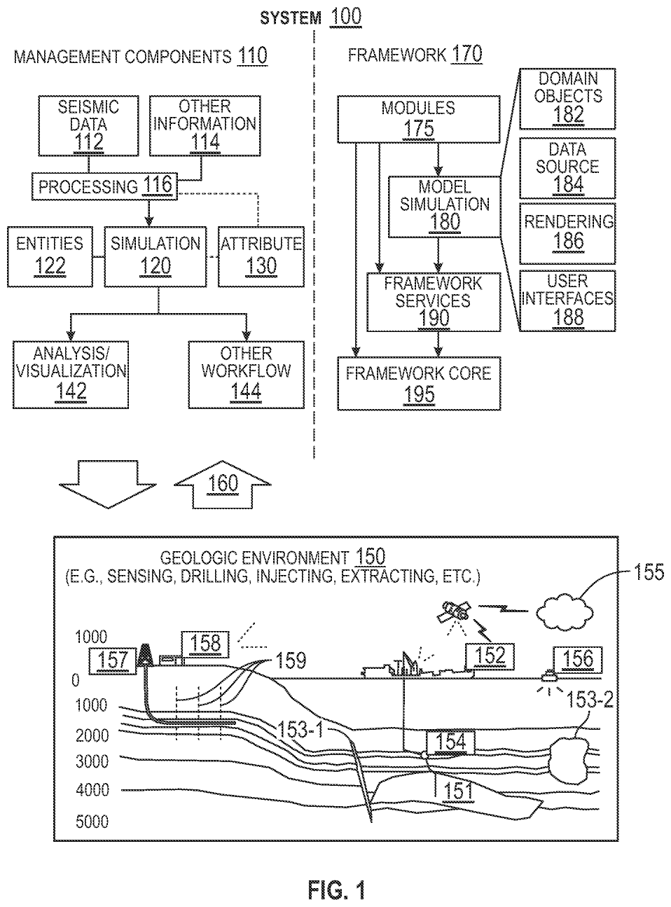

FIG. 1 illustrates an example of a system 100 that includes various management components 110 to manage various aspects of a geologic environment 150 (e.g., an environment that includes a sedimentary basin, a reservoir 151, one or more faults 153-1, one or more geobodies 153-2, etc.). For example, the management components 110 may allow for direct or indirect management of sensing, drilling, injecting, extracting, etc., with respect to the geologic environment 150. In turn, further information about the geologic environment 150 may become available as feedback 160 (e.g., optionally as input to one or more of the management components 110).

In the example of FIG. 1, the management components 110 include a seismic data component 112, an additional information component 114 (e.g., well/logging data), a processing component 116, a simulation component 120, an attribute component 130, an analysis/visualization component 142 and a workflow component 144. In operation, seismic data and other information provided per the components 112 and 114 may be input to the simulation component 120.

In an example embodiment, the simulation component 120 may rely on entities 122. Entities 122 may include earth entities or geological objects such as wells, surfaces, bodies, reservoirs, etc. In the system 100, the entities 122 can include virtual representations of actual physical entities that are reconstructed for purposes of simulation. The entities 122 may include entities based on data acquired via sensing, observation, etc. (e.g., the seismic data 112 and other information 114). An entity may be characterized by one or more properties (e.g., a geometrical pillar grid entity of an earth model may be characterized by a porosity property). Such properties may represent one or more measurements (e.g., acquired data), calculations, etc.

In an example embodiment, the simulation component 120 may operate in conjunction with a software framework such as an object-based framework. In such a framework, entities may include entities based on pre-defined classes to facilitate modeling and simulation. A commercially available example of an object-based framework is the MICROSOFT® .NET® framework (Redmond, Washington), which provides a set of extensible object classes. In the .NET® framework, an object class encapsulates a module of reusable code and associated data structures. Object classes can be used to instantiate object instances for use in by a program, script, etc. For example, borehole classes may define objects for representing boreholes based on well data.

In the example of FIG. 1, the simulation component 120 may process information to conform to one or more attributes specified by the attribute component 130, which may include a library of attributes. Such processing may occur prior to input to the simulation component 120 (e.g., consider the processing component 116). As an example, the simulation component 120 may perform operations on input information based on one or more attributes specified by the attribute component 130. In an example embodiment, the simulation component 120 may construct one or more models of the geologic environment 150, which may be relied on to simulate behavior of the geologic environment 150 (e.g., responsive to one or more acts, whether natural or artificial). In the example of FIG. 1, the analysis/visualization component 142 may allow for interaction with a model or model-based results (e.g., simulation results, etc.). As an example, output from the simulation component 120 may be input to one or more other workflows, as indicated by a workflow component 144.

As an example, the simulation component 120 may include one or more features of a simulator such as the ECLIPSE™ reservoir simulator (Schlumberger Limited, Houston Texas), the INTERSECT™ reservoir simulator (Schlumberger Limited, Houston Texas), etc. As an example, a simulation component, a simulator, etc. may include features to implement one or more meshless techniques (e.g., to solve one or more equations, etc.). As an example, a reservoir or reservoirs may be simulated with respect to one or more enhanced recovery techniques (e.g., consider a thermal process such as SAGD, etc.).

In an example embodiment, the management components 110 may include features of a commercially available framework such as the PETREL® seismic to simulation software framework (Schlumberger Limited, Houston, Texas). The PETREL® framework provides components that allow for optimization of exploration and development operations. The PETREL® framework includes seismic to simulation software components that can output information for use in increasing reservoir performance, for example, by improving asset team productivity. Through use of such a framework, various professionals (e.g., geophysicists, geologists, and reservoir engineers) can develop collaborative workflows and integrate operations to streamline processes. Such a framework may be considered an application and may be considered a data-driven application (e.g., where data is input for purposes of modeling, simulating, etc.).

In an example embodiment, various aspects of the management components 110 may include add-ons or plug-ins that operate according to specifications of a framework environment. For example, a commercially available framework environment marketed as the OCEAN® framework environment (Schlumberger Limited, Houston, Texas) allows for integration of add-ons (or plug-ins) into a PETREL® framework workflow. The OCEAN® framework environment leverages .NET® tools (Microsoft Corporation, Redmond, Washington) and offers stable, user-friendly interfaces for efficient development. In an example embodiment, various components may be implemented as add-ons (or plug-ins) that conform to and operate according to specifications of a framework environment (e.g., according to application programming interface (API) specifications, etc.).

FIG. 1 also shows an example of a framework 170 that includes a model simulation layer 180 along with a framework services layer 190, a framework core layer 195 and a modules layer 175. The framework 170 may include the commercially available OCEAN® framework where the model simulation layer 180 is the commercially available PETREL® model-centric software package that hosts OCEAN® framework applications. In an example embodiment, the PETREL® software may be considered a data-driven application. The PETREL® software can include a framework for model building and visualization.

As an example, a framework may include features for implementing one or more mesh generation techniques. For example, a framework may include an input component for receipt of information from interpretation of seismic data, one or more attributes based at least in part on seismic data, log data, image data, etc. Such a framework may include a mesh generation component that processes input information, optionally in conjunction with other information, to generate a mesh.

In the example of FIG. 1, the model simulation layer 180 may provide domain objects 182, act as a data source 184, provide for rendering 186 and provide for various user interfaces 188. Rendering 186 may provide a graphical environment in which applications can display their data while the user interfaces 188 may provide a common look and feel for application user interface components.

As an example, the domain objects 182 can include entity objects, property objects and optionally other objects. Entity objects may be used to geometrically represent wells, surfaces, bodies, reservoirs, etc., while property objects may be used to provide property values as well as data versions and display parameters. For example, an entity object may represent a well where a property object provides log information as well as version information and display information (e.g., to display the well as part of a model).

In the example of FIG. 1, data may be stored in one or more data sources (or data stores, generally physical data storage devices), which may be at the same or different physical sites and accessible via one or more networks. The model simulation layer 180 may be configured to model projects. As such, a particular project may be stored where stored project information may include inputs, models, results and cases. Thus, upon completion of a modeling session, a user may store a project. At a later time, the project can be accessed and restored using the model simulation layer 180, which can recreate instances of the relevant domain objects.

In the example of FIG. 1, the geologic environment 150 may include layers (e.g., stratification) that include a reservoir 151 and one or more other features such as the fault 153-1, the geobody 153-2, etc. As an example, the geologic environment 150 may be outfitted with any of a variety of sensors, detectors, actuators, etc. For example, equipment 152 may include communication circuitry to receive and to transmit information with respect to one or more networks 155. Such information may include information associated with downhole equipment 154, which may be equipment to acquire information, to assist with resource recovery, etc. Other equipment 156 may be located remote from a well site and include sensing, detecting, emitting or other circuitry. Such equipment may include storage and communication circuitry to store and to communicate data, instructions, etc. As an example, one or more satellites may be provided for purposes of communications, data acquisition, etc. For example, FIG. 1 shows a satellite in communication with the network 155 that may be configured for communications, noting that the satellite may additionally or instead include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

As mentioned, the system 100 may be used to perform one or more workflows. A workflow may be a process that includes a number of worksteps. A workstep may operate on data, for example, to create new data, to update existing data, etc. As an example, a may operate on one or more inputs and create one or more results, for example, based on one or more algorithms. As an example, a system may include a workflow editor for creation, editing, executing, etc. of a workflow. In such an example, the workflow editor may provide for selection of one or more pre-defined worksteps, one or more customized worksteps, etc. As an example, a workflow may be a workflow implementable in the PETREL® software, for example, that operates on seismic data, seismic attribute(s), etc. As an example, a workflow may be a process implementable in the OCEAN® framework. As an example, a workflow may include one or more worksteps that access a module such as a plug-in (e.g., external executable code, etc.).

### Volumetric Geometry Quantification of Near-Surface Carbon Containers

Embodiments of the present disclosure may facilitate quantifying storage capacity of terrestrial carbon reservoirs. Embodiments may be implemented as part of a system and method to remove anthropogenic greenhouse gas (GHG), remediating the use of fossil fuels to meet the worlds energy and product manufacturing standards.

In some embodiments, systems and methods of the present disclosure may define 2D and 3D geo-locations suitable for near surface carbon storage containment and permanent CO2 retirement. Such systems and methods may identify near-surface geological areas considered to be “nature-based natural carbon containers.” Various explorational methods may be implemented to determine and map prospective sub-surface carbon containers based on an analysis of different data sets acquired by above ground surface sensors. The analysis determines where subsurface geological settings reside that are suitable for permanent retirement of sequestered CO2 anthropogenic gases. The analysis herein uses dataset extraction methods to determine the probabilities of subsurface geological areas that may be used for carbon containment.

In some embodiments, the present disclosure provides a technical process used to set up a baseline to quantify the changes in a shallow container's properties and determine its carbon content over time. The process may use a combination of public and private historical data, field sampling, soil core analysis, and repeated 2D/3D measured profiles to build the technical datasets used by system processing and analysis tools to determine the likelihood of subsurface CO2 storage container capacity.

Such methods may also include accurately defining one or more near-surface 3D carbon storage containers, mapping the volumetric geometry of the containers, distributing the containers effective properties in a 3D grid that enables further numerical simulation and further understanding of carbon fluxes and interactions across depths. The methods can also acquire data to calibrate the 3D model and further define the sensitivity of geophysical, geochemical, and geotechnical subsurface properties.

Modeling and measurement technology may be used to estimate surface carbon aggregation over time from plant root exudates and microbial metabolism. This method may provide the capacity to map the volumetric CO2 retirement storage capacity for one or more containment locations-along with the voxelization of geophysical and/or geotechnical properties throughout a mesh and/or variable mesh compute model. The resulting output may facilitate the parameterization of reactive transport models to estimate the flux rates of topsoil carbon to the deeper near-subsurface. After carbon sinks to a depth of at least 2 meters, residency times are on the scale of hundreds to tens of thousands of years, and thus carbon at these depths may be considered “retired.”

The method may also include defining the system and process for the calibration of the data acquisition enabling further estimations for the sensitivity and/or uncertainty of different field measurements. Embodiments may also be used as reference for further practices that enable one or more methods for the acceleration of CO2 retirement within the identified storage containers, as well as methods to accurately estimate and quantify the rate of carbon sequestration.

In embodiments, methods provided herein may include using near-surface geophysical exploration data to accurately map the soil container below surface, provide a digital 3D model that can be used to understand the mechanisms of carbon sinking in terrestrial ecosystems and develop a technical protocol of setting up a baseline of CO2 charge rates, then monitor the changes over the time in 3D sense. Further, the model may be used for numerical simulation, history matching, and future prediction of CO2 changes over time.

Embodiments may account for the changes in organic carbon content in the soil container from top to bottom. In contrast, conventional methods focus on top of the soil and cannot estimate how much CO2 is lost due to farming practices and climate, and how much sinks to deeper levels.

In some embodiments, the method may receive seismic data as input. The acquired seismic data may be processed and analyzed using multi-mode surface wave analysis, modeling, and inversion (SWAMI), which produces a high-resolution velocity profile from the ground surface down to 600-1000 ft. SWAMI process is part of Omega™ seismic processing package owned by SLB.

3D modeling and calibration may be done using geological modeling package (Petrel™ in this case) that is also owned by SLB. The velocity profiles may be calibrated using electric resistivity imaging (ERI) profiles, water wells drilling reports, and regional geological and hydrological data.

Deep soil cores up to 8 ft depth may be collected and sent to industrial and academic laboratories for analyzing chemical composition, physical characteristics, and organic carbon content variation with depth. The results may be used to populate the model with attributes for further numerical simulations.

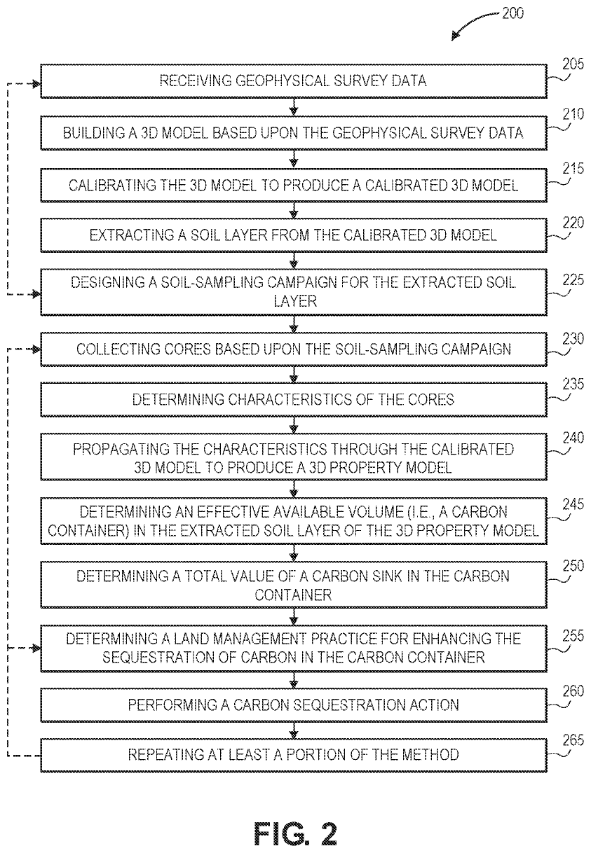

FIG. 2 illustrates a flowchart of a method 200 for carbon sequestration, according to an embodiment. At least a portion of the method 200 may be performed on a computing system. An illustrative order of the method 200 is provided below; however, one or more portions of the method 200 may be performed in a different order, simultaneously, repeated, or omitted.

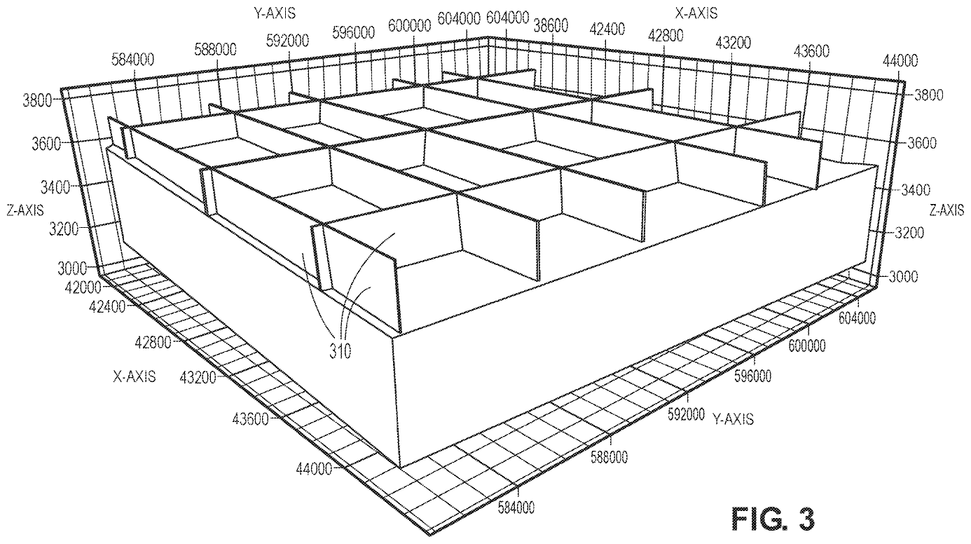

The method 200 includes receiving geophysical survey data, as at 205. The geophysical survey data may include seismic data, ground-penetrating radar, electric resistivity tomography, or a combination thereof. FIG. 3 illustrates a perspective view of the geophysical survey data, according to an embodiment. More particularly, FIG. 3 shows an orthogonal grid 310 of a geophysical profile that may be used to define a three-dimensional (3D) geo-model.

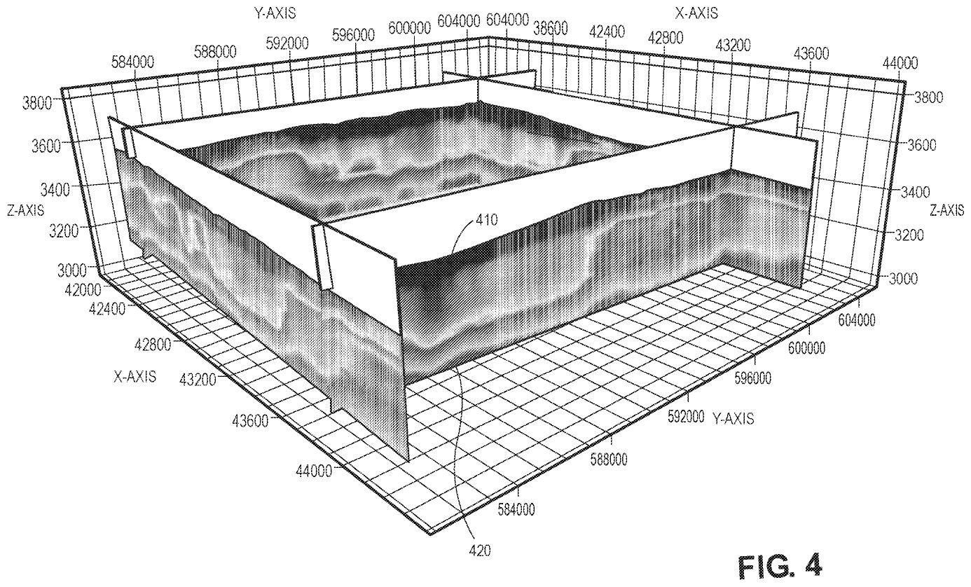

The method 200 may also include building a 3D model based upon the geophysical survey data, as at 210. The 3D model may be built using surface wave analysis modeling and inversion (SWAMI). FIG. 4 illustrates a perspective view of the 3D model, according to an embodiment. More particularly, FIG. 4 shows a processed geophysical profile that may be used to image the subsurface. The 3D model may include topsoil 410 and impermeable strata 420 and one or more layers therebetween. The topsoil 410 is above the impermeable strata 420.

The method 200 may also include calibrating the 3D model to produce a calibrated 3D model, as at 215. In an embodiment, FIG. 4 may also or instead represent the calibrated 3D model. The 3D model may be calibrated using electric resistivity tomography (ERT), ground penetrating radar (GPR), water well data, or a combination thereof.

The method 200 may also include extracting a soil layer from the calibrated 3D model, as at 220. The soil layer may include the topsoil 410 and/or the impermeable strata 420, and one or more layers therebetween.

The method 200 may also include designing a soil-sampling campaign, as at 225. The soil-sampling campaign may include identifying one or more cores in the (e.g., extracted) soil layer. More particularly, the soil sample locations (i.e., the cores) may be defined and collected based upon the review of the calibrated 3D model. The samples may be collected in locations where the calibrated 3D model indicates that the soil has (e.g., a predetermined and/or maximum) potential storage capacity. The number of cores to be collected may be defined based upon acreage in program.

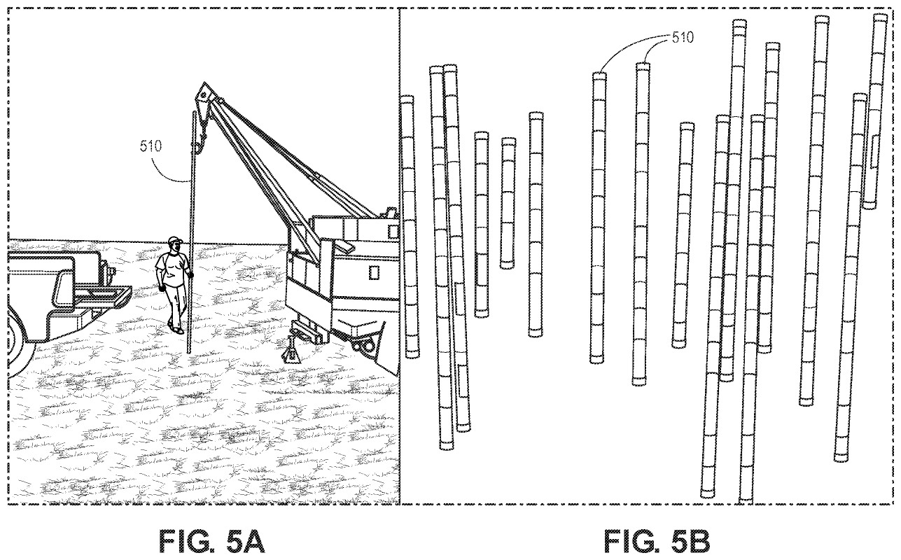

The method 200 may also include collecting the cores, as at 230. The cores may be collected based upon the soil-sampling campaign. The collection may be physical in a real-world environment (e.g., physical cores may be collected from real-world subsurface that corresponds to the extracted soil layer). FIG. 5A illustrates a perspective view of deep soil core samples 510 in the field, and FIG. 5B illustrates the core samples 510 after being collected, according to an embodiment.

The method 200 may also include determining characteristics of the cores, as at 235. More particularly, this may include determining characteristics of the (e.g., physical) cores that are collected. The characteristics may be or include biochemical soil characteristics and/or physical soil characteristics. The biochemical soil characteristics may be or include macro nutrient characteristics, micro soil nutrient characteristics, existing soil organic matter, or a combination thereof. The physical soil characteristics may be or include a density of the soil.

The method 200 may also include propagating the characteristics through the calibrated 3D model to produce a 3D property model, as at 240.

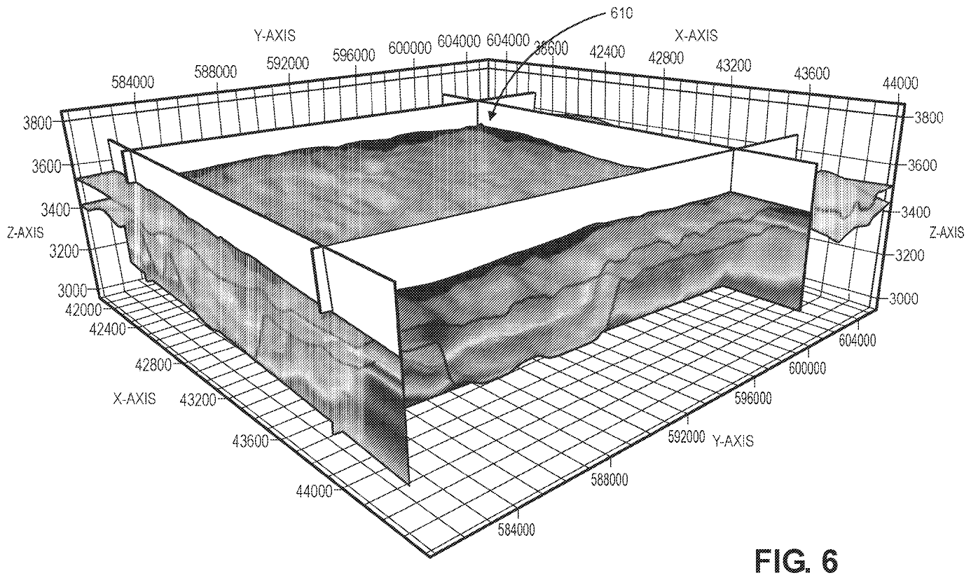

The method 200 may also include determining an effective available volume in the soil layer of the 3D property model, as at 245. The effective available volume may be referred to as a carbon container. The effective available volume may be determined based at least partially upon the 3D property model. FIG. 6 illustrates a perspective view of the effective available volume (i.e., the potential carbon storage container) 610 in the 3D property model, according to an embodiment. More particularly, FIG. 5 shows the 3D geo-model showing a potential carbon storage container.

The method 200 may also include determining a total value of a carbon sink in the carbon container, as at 250. In an example, the total value may be in grams/meters³ (g/m³). In an example, to determine the total value, let f(X) be the relative local density of soil, and g (X) be the corresponding local average granule size. The variable g(X) may be approximated with the sphere packing of spheres of radius equal to half the average mean granule dimension, \hat{ }g(X). Hence, the local axial porosity may be given by:

$$ϵ ( X ) = 1 - {}^{\hat{}} g ( X ) / V \qquad Equation ( 1 )$$

where V is the total local volume.

Finally, the organic matter holding capacity may depend in detail on the surface chemistry of soil granules, which is obtained by mineralogical and biochemical analyses.

An equation for mineral saturation of soil granules is provided below:

$$Csat - pot = 4.09 + 0.37 * P ( Particles \leq 20 μm ) \qquad Equation ( 2 )$$

where P(·) is the fraction of particles less than 20 μm, and Cₛₐₜ₋ₚₒₜ gives the carbon saturation potential. This estimate may be augmented with mineralogical and/or chemistry information by collecting Cation Exchange Capacity (CEC) and mineralogical analyses to more accurately parameterize C_{sat pot} using a machine learning (ML) framework. Specifically, the empirical measurements of Cₛₐₜ₋ₚₒₜ may be regressed as a function of CEC, mineralogical, and other chemical covariates. The refined estimate of Cₛₐₜ₋ₚₒₜ may constitute an additive contribution to the organic matter buildup in the interstitial space, yielding a volumetric integral as follows:

$${f ( X )}^{- 1} ϵ ( X ) + {C}_{sat - pos} ( X ) dX \qquad Equation ( 3 )$$

where X indexes the spatial coordinates within the volume, accounting for spatial heterogeneity within the container. In high clay soils, the Cₛₐₜ₋ₚₒₜ contribution may be non-negligible.

The method 200 may also include determining a land management practice for enhancing the carbon sequestration in the carbon container, as at 255. The land management practice may be determined based upon the 3D property model and/or the total value of the carbon sink.

The method 200 may also include performing a carbon sequestration action, as at 260. For example, this may include enhancing the carbon sequestration in the carbon container. The carbon sequestration action may be performed based upon the 3D property model, the effective available volume, the land management practice, or a combination thereof. The carbon sequestration action may be or include generating and/or transmitting a signal (e.g., using a computing system) that causes a physical action to occur at a wellsite. The carbon sequestration action may also or instead include performing the physical action at the wellsite. The physical action may be or include sequestrating the carbon in the carbon container using the land management practice.

The method 200 may also include repeating at least a portion of the method 200, as at 265. In an example, the method 200 may loop back to 205, 225, 230, and/or 255 and repeat. The method 200 may be repeated after a predetermined amount of time to quantify a change in an amount of the carbon sequestrated (e.g., between a first iteration and a second iteration of the method 200).

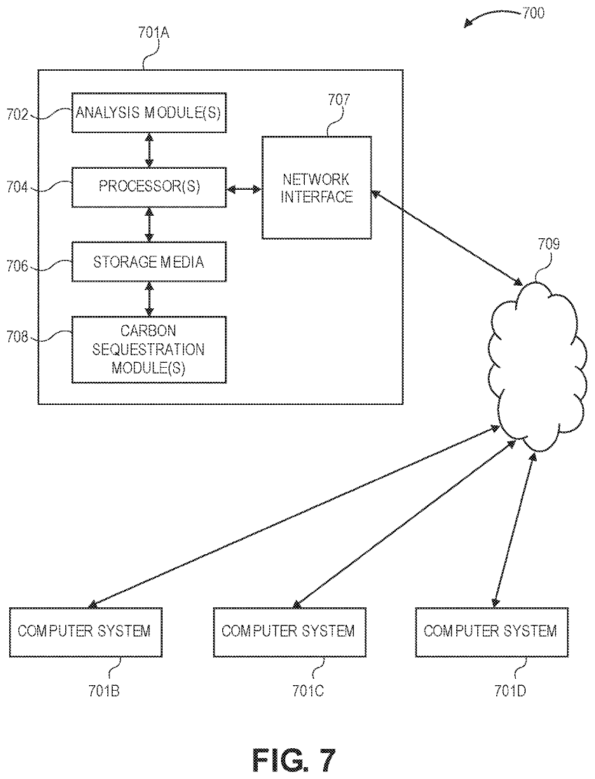

In some embodiments, the methods of the present disclosure may be executed by a computing system. FIG. 7 illustrates an example of such a computing system 700, in accordance with some embodiments. The computing system 700 may include a computer or computer system 701A, which may be an individual computer system 701A or an arrangement of distributed computer systems. The computer system 701A includes one or more analysis modules 702 that are configured to perform various tasks according to some embodiments, such as one or more methods disclosed herein. To perform these various tasks, the analysis module 702 executes independently, or in coordination with, one or more processors 704, which is (or are) connected to one or more storage media 706. The processor(s) 704 is (or are) also connected to a network interface 707 to allow the computer system 701A to communicate over a data network 709 with one or more additional computer systems and/or computing systems, such as 701B, 701C, and/or 701D (note that computer systems 701B, 701C and/or 701D may or may not share the same architecture as computer system 701A, and may be located in different physical locations, e.g., computer systems 701A and 701B may be located in a processing facility, while in communication with one or more computer systems such as 701C and/or 701D that are located in one or more data centers, and/or located in varying countries on different continents).

A processor may include a microprocessor, microcontroller, processor module or subsystem, programmable integrated circuit, programmable gate array, or another control or computing device.

The storage media 706 may be implemented as one or more computer-readable or machine-readable storage media. Note that while in the example embodiment of FIG. 7 storage media 706 is depicted as within computer system 701A, in some embodiments, storage media 706 may be distributed within and/or across multiple internal and/or external enclosures of computing system 701A and/or additional computing systems. Storage media 706 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above may be provided on one computer-readable or machine-readable storage medium, or may be provided on multiple computer-readable or machine-readable storage media distributed in a large system having possibly plural nodes. Such computer-readable or machine-readable storage medium or media is (are) considered to be part of an article (or article of manufacture). An article or article of manufacture may refer to any manufactured single component or multiple components. The storage medium or media may be located either in the machine running the machine-readable instructions, or located at a remote site from which machine-readable instructions may be downloaded over a network for execution.

In some embodiments, computing system 700 contains one or more carbon sequestration module(s) 708. In the example of computing system 700, computer system 701A includes the carbon sequestration module 708. In some embodiments, a single carbon sequestration module may be used to perform some aspects of one or more embodiments of the methods disclosed herein. In other embodiments, a plurality of carbon sequestration modules may be used to perform some aspects of methods herein.

It should be appreciated that computing system 700 is merely one example of a computing system, and that computing system 700 may have more or fewer components than shown, may combine additional components not depicted in the example embodiment of FIG. 7, and/or computing system 700 may have a different configuration or arrangement of the components depicted in FIG. 7. The various components shown in FIG. 7 may be implemented in hardware, software, or a combination of both hardware and software, including one or more signal processing and/or application specific integrated circuits.

Further, the steps in the processing methods described herein may be implemented by running one or more functional modules in information processing apparatus such as general purpose processors or application specific chips, such as ASICs, FPGAs, PLDs, or other appropriate devices. These modules, combinations of these modules, and/or their combination with general hardware are included within the scope of the present disclosure.

Computational interpretations, models, and/or other interpretation aids may be refined in an iterative fashion; this concept is applicable to the methods discussed herein. This may include use of feedback loops executed on an algorithmic basis, such as at a computing device (e.g., computing system 700, FIG. 7), and/or through manual control by a user who may make determinations regarding whether a given step, action, template, model, or set of curves has become sufficiently accurate for the evaluation of the subsurface three-dimensional geologic formation under consideration.

The foregoing description, for purpose of explanation, has been described with reference to specific embodiments. However, the illustrative discussions above are not intended to be exhaustive or limiting to the precise forms disclosed. Many modifications and variations are possible in view of the above teachings. Moreover, the order in which the elements of the methods described herein are illustrate and described may be re-arranged, and/or two or more elements may occur simultaneously. The embodiments were chosen and described in order to best explain the principles of the disclosure and its practical applications, to thereby enable others skilled in the art to best utilize the disclosed embodiments and various embodiments with various modifications as are suited to the particular use contemplated.

What is claimed is:

1. A method for carbon sequestration, the method comprising:
receiving geophysical survey data;
building a three-dimensional (3D) model based upon the geophysical survey data;
calibrating the 3D model to produce a calibrated 3D model;
extracting a soil layer from the calibrated 3D model to produce an extracted soil layer;
designing a soil-sampling campaign based upon the extracted soil layer, wherein the soil-sampling campaign comprises a plurality of cores;
determining characteristics of the cores; and
propagating the characteristics through the calibrated 3D model to produce a 3D property model.

2. The method of claim 1, wherein the geophysical survey data comprises seismic data, ground-penetrating radar, electric resistivity tomography, or a combination thereof.

3. The method of claim 1, wherein the 3D model is built using surface wave analysis modeling and inversion (SWAMI), wherein the 3D model comprises topsoil and impermeable strata, and wherein the topsoil is above the impermeable strata.

4. The method of claim 1, wherein the 3D model is calibrated using electric resistivity tomography (ERT), ground penetrating radar (GPR), water well data, or a combination thereof.

5. The method of claim 1, wherein the soil layer comprises topsoil down to an impermeable strata.

6. The method of claim 1, further comprising receiving physical cores from a real-world subsurface, wherein the physical cores correspond to the cores in the extracted soil layer of the calibrated 3D model, and wherein the characteristics are determined from the physical cores.

7. The method of claim 1, wherein the characteristics comprise biochemical soil characteristics and physical soil characteristics.

8. The method of claim 7, wherein the biochemical soil characteristics comprise macro nutrient characteristics, micro soil nutrient characteristics, existing soil organic matter, or a combination thereof, and wherein the physical soil characteristics comprise a density of the soil.

9. The method of claim 1, further comprising determining an effective available volume in the soil layer of the 3D property model, wherein the effective available volume is a carbon container.

10. The method of claim 9, further comprising:
determining a total value of a carbon sink in the carbon container based upon the effective available volume; and
sequestrating carbon in the carbon container based upon the total value of the carbon sink.

11. A computing system, comprising:
one or more processors; and
a memory system comprising one or more non-transitory computer-readable media storing instructions that, when executed by at least one of the one or more processors, cause the computing system to perform operations, the operations comprising:
receiving geophysical survey data, wherein the geophysical survey data comprises seismic data, ground-penetrating radar, electric resistivity tomography, or a combination thereof;
building a three-dimensional (3D) model based upon the geophysical survey data, wherein the 3D model is built using surface wave analysis modeling and inversion (SWAMI), wherein the 3D model comprises topsoil and impermeable strata, and wherein the topsoil is above the impermeable strata;
calibrating the 3D model to produce a calibrated 3D model, wherein the 3D model is calibrated using electric resistivity tomography (ERT), ground penetrating radar (GPR), water well data, or a combination thereof;
extracting a soil layer from the calibrated 3D model to produce an extracted soil layer, wherein the soil layer comprises the topsoil and the impermeable strata;
designing a soil-sampling campaign based upon the extracted soil layer, wherein the soil-sampling campaign comprises a plurality of cores;
determining characteristics of physical cores from a real-world subsurface, wherein the physical cores correspond to the cores in the extracted soil layer of the calibrated 3D model, wherein the characteristics comprise biochemical soil characteristics and physical soil characteristics, wherein the biochemical soil characteristics comprise macro nutrient characteristics, micro soil nutrient characteristics, existing soil organic matter, or a combination thereof, and wherein the physical soil characteristics comprise a density of the soil; and
propagating the characteristics through the calibrated 3D model to produce a 3D property model.

12. The computing system of claim 11, wherein the operations further comprise determining an effective available volume in the extracted soil layer of the 3D property model, wherein the effective available volume is a carbon container.

13. The computing system of claim 12, wherein the operations further comprise determining a total value of a carbon sink in the carbon container, and wherein the total value is determined based at least partially upon the effective available volume.

14. The computing system of claim 13, wherein the operations further comprise determining a land management practice for sequestrating carbon in the carbon container, and wherein the land management practice is determined based at least partially upon the total value of the carbon sink.

15. The computing system of claim 14, wherein the operations further comprise generating or transmitting a signal that causes the carbon to be sequestrated in the carbon container using the land management practice.

16. A non-transitory computer-readable medium storing instructions that, when executed by one or more processors of a computing system, cause the computing system to perform operations, the operations comprising:
receiving geophysical survey data, wherein the geophysical survey data comprises seismic data, ground-penetrating radar, and electric resistivity tomography;
building a three-dimensional (3D) model based upon the geophysical survey data, wherein the 3D model is built using surface wave analysis modeling and inversion (SWAMI), wherein the 3D model comprises topsoil and impermeable strata, and wherein the topsoil is above the impermeable strata;
calibrating the 3D model to produce a calibrated 3D model, wherein the 3D model is calibrated using electric resistivity tomography (ERT), ground penetrating radar (GPR), and water well data;
extracting a soil layer from the calibrated 3D model to produce an extracted soil layer, wherein the extracted soil layer comprises the topsoil and the impermeable strata;
designing a soil-sampling campaign based upon the extracted soil layer, wherein the soil-sampling campaign comprises a plurality of physical cores to be collected from a real-world subsurface;
determining characteristics of the physical cores, wherein the characteristics comprise biochemical soil characteristics and physical soil characteristics, wherein the biochemical soil characteristics comprise macro nutrient characteristics, micro soil nutrient characteristics, and existing soil organic matter, and wherein the physical soil characteristics comprise a density of the soil:
propagating the characteristics through the calibrated 3D model to produce a 3D property model;
determining an effective available volume in the extracted soil layer of the 3D property model, wherein the effective available volume is a carbon container;
determining a total value of a carbon sink in the carbon container, wherein the total value is determined based upon the effective available volume; and
determining a land management practice for sequestrating carbon in the carbon container, wherein the land management practice is determined based upon the 3D property model and the total value of the carbon sink.

17. The non-transitory computer-readable medium of claim 16, wherein the total value of the carbon sink in the carbon container is determined using:
$${f ( X )}^{- 1} ϵ ( X ) + {C}_{sat - pos} ( X ) dX$$
where f(X) is the density of the soil, and Cₛₐₜ-Cₚₒₜ is a carbon saturation potential.

18. The non-transitory computer-readable medium of claim 16, wherein the operations further comprise generating or transmitting a signal to cause a carbon sequestration action to be performed based at least partially upon the total value of the carbon sink, the land management practice, or both.

19. The non-transitory computer-readable medium of claim 18, wherein the carbon sequestration action comprises physically sequestrating the carbon in the carbon container using the land management practice.

20. The non-transitory computer-readable medium of claim 19, wherein the operations further comprise repeating at least a portion of the operations after a predetermined amount of time to quantify a change in an amount of the carbon sequestrated in the carbon container.
